(19) United States
(12) Patent Application Publication
(10) Pub. No.: US 2018/0368815 A1
(43) Pub. Date: Dec. 27, 2018

(54) BODY FLUID STATE DIAGNOSTIC SYSTEM AND STATE DIAGNOSTIC METHOD USING SAME

(71) Applicant: JONGROMEDICAL CO., LTD., Daegu (KR)

(72) Inventor: Ji Hoon KIM, Daegu (KR)

(73) Assignee: JONGROMEDICAL CO., LTD., Daegu (KR)

(21) Appl. No.: 16/062,631

(22) PCT Filed: Nov. 22, 2016
(86) PCT No.: PCT/KR2016/013459

§ 371 Date: Jun. 15, 2018

(30) Foreign Application Priority Data
Dec. 15, 2015 (KR) 10-2015-0179431

(51) Int. Cl. A61B 10/00; G06T 7/00; G16H 30/40

(52) U.S. Cl. CPC A61B 10/0012; A61B 10/0051; G16H 80/00; G16H 30/40; A61B 2010/009; G06T 7/0012

(57) ABSTRACT

The present invention relates to a body fluid state diagnostic system and body fluid state diagnostic method using the same, the system and method enabling a user to perform self-diagnosis and identify the diagnostic result as well as enabling reconfirmation and a diagnosis through a specialized medical institution, whereby the user may quickly and accurately identify the body state of the user, and may conveniently access specialized medical technologies of the specialized medical institution.

### TECHNICAL FIELD

The present invention relates to a body fluid state diagnostic system and method. More particularly, the present invention relates to a body fluid state diagnostic system and body fluid state diagnostic method using the same, the system and method enabling a user to perform self-diagnosis and identify the diagnostic result as well as enabling reconfirmation and a diagnosis through a specialized medical institution, whereby the user may quickly and accurately identify the body state of the user, and may conveniently access specialized medical technologies of the specialized medical institution.

### BACKGROUND ART

Generally, people should visit a specialized medical institution to check there health state and body state. However, in order to receive diagnosis, people need to set aside time for visiting a specialized medical institution during busy work time, and it takes much time and money to perform the complicated procedure. Thus, most people ignore endurable discomfort, and visit specialized medical institutions only when having unendurable pain or discomfort.

In order to solve the inconvenience, techniques have been developed wherein various types of advanced equipment are used to frequently check one's body state and the result is transmitted to a specialized medical institution via a network. However, techniques that are commercialized and are actually used in practice are extremely rare.

Preferably, the use of a simple tester, which has been used for a long time, continues and is increasingly used by the younger generation due to ease of use, low cost, and convenience, and thus visiting a specialized medical institution is unnecessary.

Such a tester detects biochemical substances such as hormones discharged under particular circumstances by using secretions from people's bodies, e.g., saliva, urine, sweat, etc. The tester is relatively highly accurate and easy to use. Accordingly, as a commonly used testing means, testers for pregnancy testing, diabetes diagnosis, and blood sugar checking are used.

However, the testers are limited in use and are incapable of providing more information to the user. Particularly, the testers are disposable, which are thrown away after the user checks, and thus a diagnosis in connection to the specialized medical institution is difficult.

That is, conventional testers provide convenience of only use but lack integration with existing medical service.

### DISCLOSURE

### Technical Problem

Accordingly, the body fluid state diagnostic system and body fluid state diagnostic method using the same enable the user to perform self-diagnosis and identify the diagnostic result as well as enables reconfirmation and diagnosis through a specialized medical institution, whereby the user may quickly and accurately identify the body state of the user, and may conveniently access specialized medical technologies of the specialized medical institution.

Particularly, the body fluid state diagnostic system and body fluid state diagnostic method using the same enable the user wishing to become pregnant, to directly identify the menstrual cycle and to identify sperm activity of the user so as to check whether there is abnormality and a diagnosis of the specialized medical institution is necessary, thereby helping the user to become pregnant at a desired time. Also, it is possible to know whether a visit to a specialized medical institution is necessary such that inconvenience of visiting the specialized medical institution avoided.

### Technical Solution

In order to accomplish the above object, according to the present invention, there is provided a body fluid state diagnostic system including: a testing tool having a base on which a test target that is body fluid of a user is deposited; and a user terminal obtaining a test image of the body fluid deposited on the base by photographing the base of the testing tool, generating analysis information by analyzing the test image, and outputting the analysis information depending on selection of the user.

The system may further include a medical institution system receiving the test image or the analysis information via a network in connection to the user terminal, storing the test image or the analysis information by matching with information of the user which is pre-stored, and performing a reanalysis by using the test image to determine validity of the analysis information or to provide an analysis result to a medical specialist.

The body fluid may be saliva, semen, or vaginal discharge of the user.

The user terminal may perform a pattern analysis of a crystalline characteristic including at least one of density, a direction, and a length of a crystal formed from the body fluid in the test image.

The user terminal may perform a pattern analysis of a sperm characteristic including at least one of activity, density, and malformation of a sperm in the body fluid.

The activity of the sperm may include at least one of straightness, a straight-line distance, and a rotational tendency of the sperm.

The testing tool or the user terminal or the medical institution system may use a subdivision pattern limiting a range of the image to determine the crystalline characteristic or the sperm characteristic.

The user terminal or the medical institution system may perform the pattern analysis on a target which is the crystal formed within the subdivision pattern or the sperm positioned within the subdivision pattern.

The testing tool may be detachably attached to the user terminal.

The testing tool may include an enlargement unit providing an enlarged image of the body fluid deposited on the base.

The user terminal may photograph the body fluid provided by the testing tool in a magnified manner.

Also, according to the present invention, there is provided a body fluid state diagnostic method including: a body fluid reception step at which body fluid of a user is deposited on a base of a testing tool; an image acquisition step at which a user terminal photographs the base and obtains a test image of the body fluid; an analysis information generation step at which the user terminal analyzes the test image to generate analysis information; and an output step at which the user terminal outputs the analysis information.

The method may further include: a transmission step at which the user terminal is connected to a medical institution system of a specialized medical institution via a network and the test image or the analysis information is transmitted to the medical institution system; a storage step at which the medical institution system stores the analysis information or the test image by matching with information of the user; and a diagnosis step at which the medical institution system transmits the analysis information to a medical specialist of the specialized medical institution.

The storage step or the diagnosis step may include a regeneration step at which the medical institution system performs a reanalysis of the test image to regenerate the analysis information.

The body fluid may be saliva, semen, or vaginal discharge of the user.

At the analysis information generation step or the regeneration step, the user terminal or the medical institution system may perform, by using the test image, a pattern analysis of a crystalline characteristic including at least one of density, a direction, and a length of a crystal formed from the body fluid.

At the analysis information generation step or the regeneration step, the user terminal or the medical institution system may perform a pattern analysis of a sperm characteristic including at least one of straightness, a straight-line distance, and a rotational tendency of a sperm.

At one of the image acquisition step, the analysis information generation step, and the regeneration step, the testing tool or the user terminal or the medical institution system may use a subdivision pattern limiting a range of the image to determine the crystalline characteristic or the sperm characteristic of the body fluid.

At one of the image acquisition step, the analysis information generation step, and the regeneration step, the pattern analysis may be performed on a target which is the crystal formed within the subdivision pattern or the sperm positioned within the subdivision pattern.

At the image acquisition step, the user terminal may photograph the body fluid in a magnified manner.

### Advantageous Effects

According to the present invention, the body fluid state diagnostic system and body fluid state diagnostic method using the same enable the user to perform self-diagnosis and identify the diagnostic result as well as enable reconfirmation and a diagnosis through the specialized medical institution, whereby the user may quickly and accurately identify the body state of the user, and may conveniently access specialized medical technologies of the specialized medical institution.

According to the present invention, the body fluid state diagnostic system and body fluid state diagnostic method using the same enable the user wishing to become pregnant, to directly identify the menstrual cycle and to identify sperm activity of the user so as to check whether there is abnormality and a diagnosis of the specialized medical institution is necessary, thereby helping the user to become pregnant at a desired time. Also, it is possible to know whether a visit to the specialized medical institution is necessary such that inconvenience of visiting the specialized medical institution avoided.

### DESCRIPTION OF DRAWINGS

FIG. 1 is a diagram illustrating an example of configuration of a body fluid state diagnostic system according to the present invention.

FIG. 2 is a diagram illustrating an example of a testing tool in FIG. 1.

FIG. 3 is a diagram illustrating an example of a change in body fluid for a pattern analysis.

FIG. 4 is a diagram illustrating an example of a test image in which actual body fluid is used.

FIG. 5 is a diagram illustrating an example of a subdivision pattern used in analyzing a test image.

FIG. 6 is a diagram illustrating an example of a case when body fluid is semen and vaginal discharge.

FIG. 7 is a flowchart illustrating a body fluid state diagnostic method according to the present invention.

### BEST MODE

Hereinafter, exemplary embodiments of the present invention will be described with reference to the accompanying drawings. In the following description, the same elements will be designated by the same reference numerals although they are shown in different drawings. Further, in the following description of the present invention, a detailed description of known functions and configurations incorporated herein will be omitted when it may make the subject matter of the present invention rather unclear. In the drawing, certain features presented in the drawings are expanded, reduced, or simplified for easy explanation, and the drawings and the constituent elements may not be appropriately illustrated. However, those of ordinary skill in the art could easily understand such detailed matters.

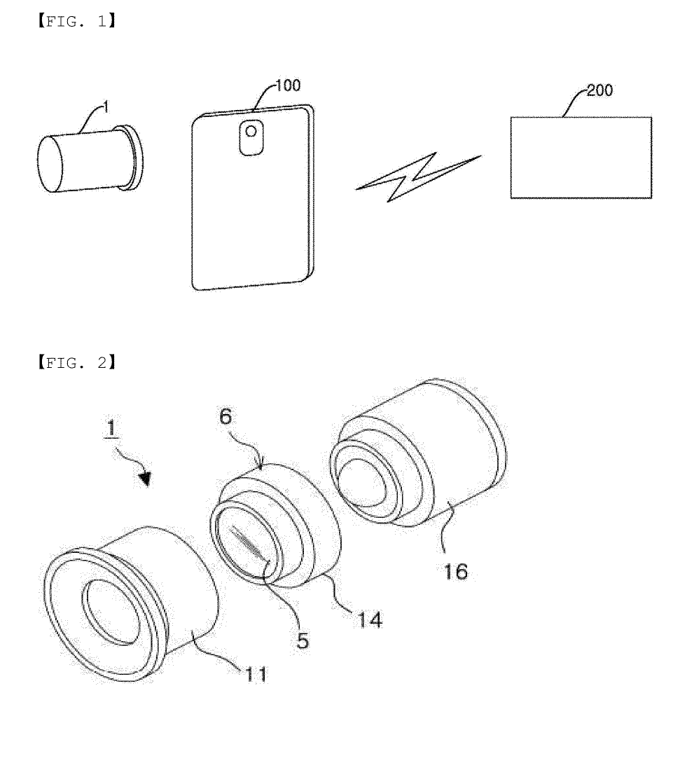

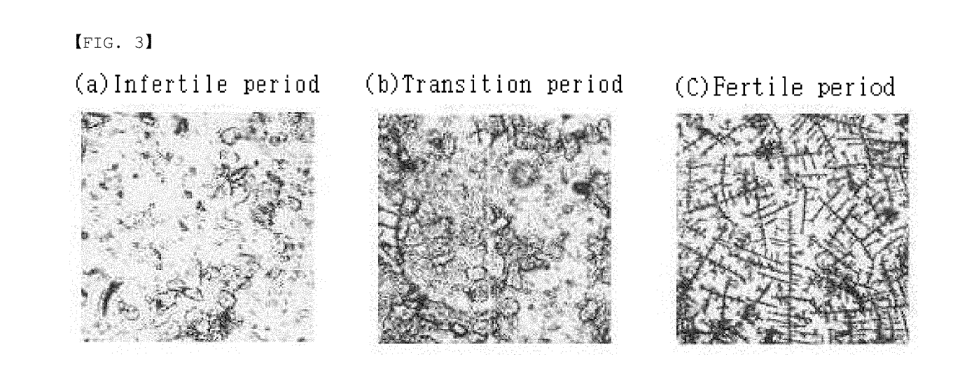

FIG. 1 is a diagram illustrating an example of configuration of a body fluid state diagnostic system according to the present invention. FIG. 2 is a diagram illustrating an example of a testing tool in FIG. 1.

Referring to FIGS. 1 and 2, according to the present invention, the body fluid state diagnostic system includes: a testing tool 1; a user terminal 100; and a medical institution system 200.

The testing tool 1 to which an analysis target, which is body fluid of the user, is deposited enables the user terminal 100 to obtain an image of the body fluid. That is, the user who wants to have an examination deposits the body fluid on a base 6 of the testing tool 1, and connects the testing tool 1 to the user terminal 100 to be in contact with each other. Then, the user terminal 100 photographs the state of the body fluid deposited on the base 6 such that a test image is obtained.

To this end, the testing tool 1 may include a contact unit 11, the base 6, and a lighting unit 16 as shown in FIG. 2.

The base 6 on which the body fluid of the user who wants to have an examination is deposited severs as a slide glass of a microscope, namely, a glass on which the target to be observed is placed and a stage fixing the glass. To this end, the base 6 includes a base body 14 and a sheet unit 5. The sheet unit 5 on which the body fluid of the user is deposited is formed of a transparent material to transmit the light supplied from the lighting unit 16. The user terminal 100 photographs the body fluid deposited on the sheet unit 5 to obtain an image thereof via the contact unit 11. The sheet unit 5 may be formed of a material, such as transparent synthetic resins, e.g., acrylic or polyethylene resin, or glass. However, the present invention is not limited thereto, and any material that is capable of transmitting light from the lighting unit 16 may be used. Also, the sheet unit 5 may be provided with a coloring or polarization function depending on the type of body fluid and a body fluid identification method.

The contact unit 11 is coupled to the photographing unit of the user terminal 100 to enable the photographing unit to obtain an image of the body fluid deposited on the base 6 without interference from external light. Particularly, the contact unit 11 serves as an objective lens containing a magnification portion, e.g., a magnifying lens group, and a body tube so as to compensate for insufficient magnification the photographing unit of the user terminal 100. Particularly, the contact unit 11 may include a coupling member for detachably attaching the testing tool 1 to the user terminal 100, but the coupling member is omitted in FIGS. 1 and 2.

The lighting unit 16 provides background light to enable the user terminal 100 to easily obtain the image of the body fluid deposited on the sheet unit 5. The lighting unit 16 may supply artificial light in direction of the contact unit 11 through the sheet unit 5, or may transmit natural light, but these do not limit the present invention.

The user terminal 100 photographs the body fluid deposited on the base of the testing tool to obtain the image of the body fluid, analyzes the obtained test image to generate analysis information, and outputs the analysis information to the user.

Specifically, the testing tool containing the base on which the body fluid is deposited is coupled to the photographing unit of the user terminal 100, particularly, in front of the lens. When the testing tool is coupled, the user terminal 110 obtains the test image by photographing the base 6, more precisely, the body fluid deposited on the base 6 identified through the contact unit 11.

The user terminal 100 performs image processing on the test image by using an internally-determined algorithm, and conducts an analysis to determine a crystalline characteristic and a sperm characteristic. Next, the user terminal 100 outputs the analysis result via an output unit of the user terminal 100.

Also, the user terminal 100 transmits the analysis result to the medical institution system 200 of the specialized medical institution. To this end, the user terminal 100 may access a mobile network and a wired or wireless data network for connection to the medical institution system 200, and may include a communication module (not shown) therefor.

The user terminal 100 may be a dedicated terminal for the test, or may be configured by a mobile terminal, such as a smart phone, a cellular phone, and a tablet that are generally used, and by an app (or application) installed on the mobile terminal.

The medical institution system 200 is connected to the user terminal 100 via a network, and receives the test image and the analysis information from the user terminal 100 to store them by matching with user information. Also, the medical institution system conducts a reanalysis using the test image, and checks the validity of the analysis information or provides the analysis information to a medical specialist.

The medical institution system 200 may be a system of a hospital, or may be an intermediate system between the hospital and the user. Particularly, the medical institution system 200 conducts a reanalysis of the test image such that whether the analysis information generated by the user terminal 100 is valid is reconfirmed. In this process, various analyzes that are unable to be executed in the user terminal 100 may be conducted. Particularly, the medical institution system 200 provides the analysis result to a medical specialist, such as a doctor, so as to check whether there is malformation and whether a diagnosis is necessary according to the analysis information.

Furthermore, the medical institution system 200 may transmit the diagnostic result from the medical specialist to the user terminal 100, or may transmit a request from the medical specialist to the user terminal 100 for a diagnosis. To this end, the medical institution system 200 may include: an information storage unit receiving and storing the user information, the analysis information, and the test image; an analysis unit conducting verification of the analysis information or an analysis of the test image; and a communication unit connecting the medical specialist and the user terminal.

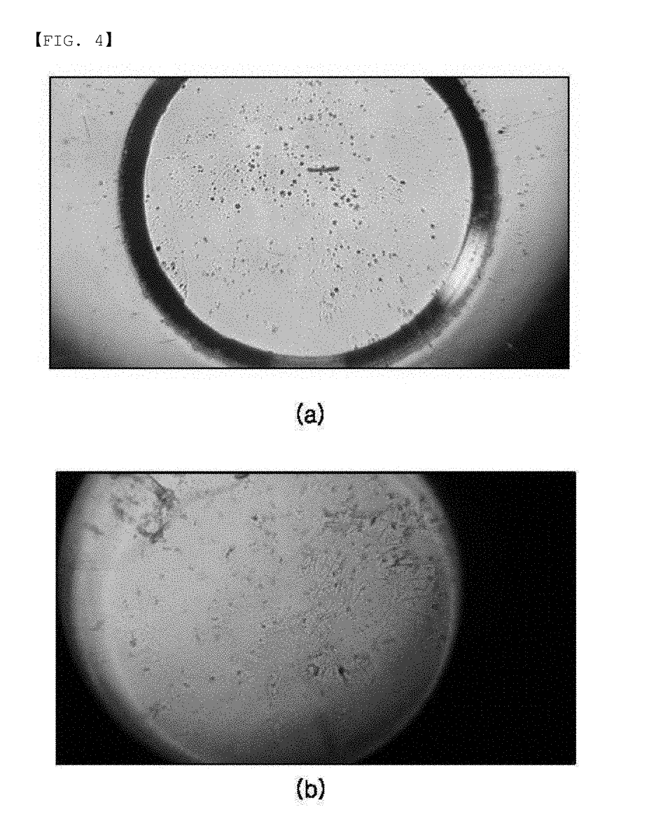

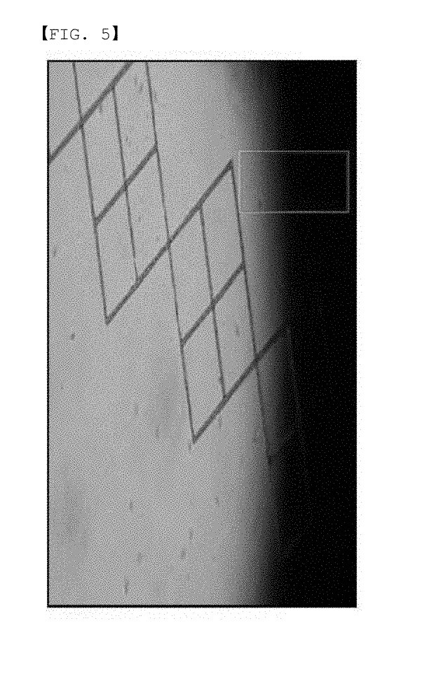

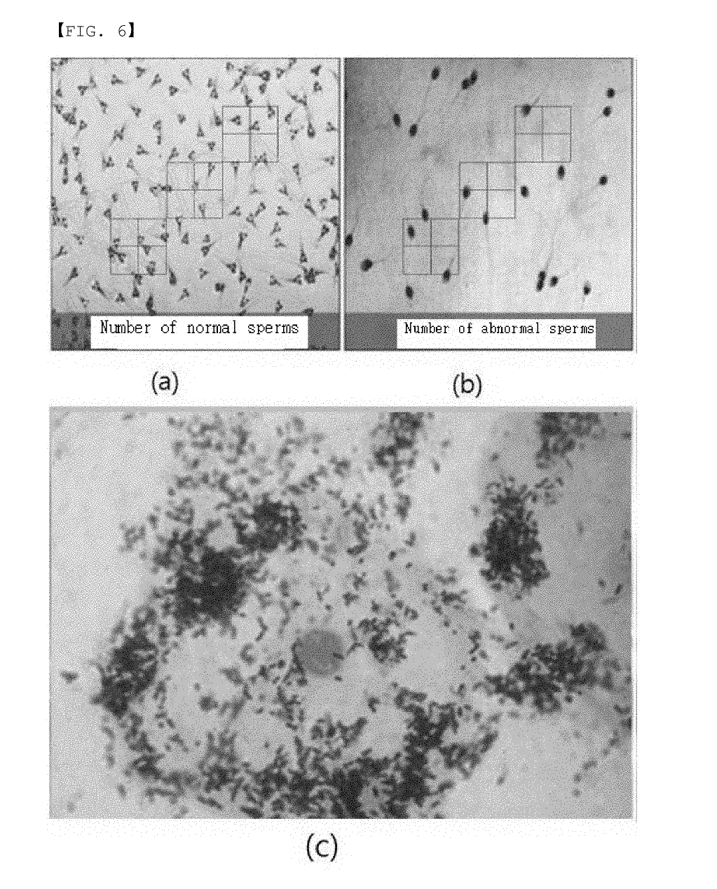

FIGS. 3 to 5 are diagrams illustrating an example of a pattern analysis method according to the present invention. FIG. 3 is a diagram illustrating an example of a change in body fluid for a pattern analysis. FIG. 4 is a diagram illustrating an example of a test image in which actual body fluid is used. Also, FIG. 5 is a diagram illustrating an example of a subdivision pattern used in analyzing a test image.

Referring to FIGS. 3 to 5, the body fluid, particularly, the saliva may be used as an indicator of various conditions. The saliva of a woman in the fertile period shows a special sign distinctly. Specifically, due to the influence of estrogen which is a female hormone contained in the saliva of a woman, ovulation may be accurately predicted, and thus married couples suffering from infertility may increase pregnancy probability.

Specifically, when a woman in the fertile period, as the ovulation date gets closer, the secretion rate of the estrogen increases, and also, the salt concentration of the saliva increases. Accordingly, when the saliva is dried for a particular time, different crystal structures of the dried saliva shows depending on the infertile period, the transition period, and the fertile period.

For example, when drying and photographing the saliva of the woman in the infertile period during which ovulation does not occur, an image in which no distinct crystal structure is found is obtained as shown in FIG. 3(a).

FIG. 4(a) shows the actually obtained image. As shown in FIGS. 3(a) and 4(a), in the infertile period, no specific crystal structure is found, and only some salivary cells are identified.

In the transition period during which the infertile period transitions to the fertile period in which ovulation occurs, the saliva of the woman is slightly crystallized in the form as shown in FIG. 3(b). At this time, the form of the crystals is the form of salivary cells in circular and oblong shapes formed by electrolyte, mucus, enzyme, etc.

Last, in the fertile period during which ovulation occurs, the form in which the crystal extends in one direction, namely, a fern pattern is clearly visible as shown in FIG. 3(c). FIG. 4(b) shows an image of actual saliva of the woman in the fertile period. As seen from this, the pattern distinctly distinguished from FIG. 4(a) is identified.

In the body fluid state diagnostic system of the present invention, the state of the saliva is analyzed to identify whether the user is in the fertile period, and the identified information is provided to the user as the analysis information.

That is, the user terminal 100 performs image processing on the obtained test image to clarify the crystal line, and determines whether the crystal line is in the form of a closed curve line or in the form of the straight lines in the fern pattern as shown in FIG. 3(c). Particularly, the length and density of the linear pattern is identified. Consequently, the beginning of the ovulation period or of the fertile period, the certainty period, the transition period, and the infertile period are determined and the result is provided to the user as the analysis information. Here, the image processing and the determining of the beginning, the transition period, the certainty period, and the infertile period are possibly adjusted depending on the density and form of the pattern, and thus the detailed description thereof will be omitted.

Also, in image processing, the image is reprocessed in black and white, and the contrast of black and white is distinguished to determine the form of the line, whereby the length and the density is determined. Since various methods may be used, the detailed description thereof will be omitted.

In the meantime, in order to enhance accuracy of image processing and the determination, a subdivision pattern may be used as shown in FIG. 5. That is, how many straight lines or oval lines are present in the region (the region inside the closed line) defined by the pattern is identified, and the analysis information is generated.

Particularly, the subdivision pattern may be provided in a printed form on the lens of the testing tool 1 or the sheet unit 5 of the base 6, or a part of the analysis image may be partitioned in the subdivision pattern by the user terminal for image processing, but the present invention is not limited thereto.

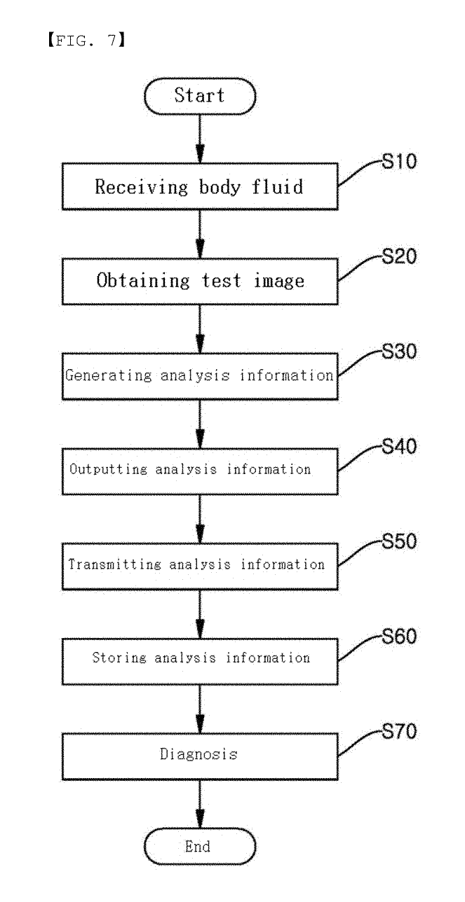

In the meantime, FIG. 6 is a diagram illustrating an example of a case when body fluid is semen and vaginal discharge.

The analysis information may be generated with respect to semen of a man. When using the state diagnostic system of the present invention with respect to semen of a man, matters that the number of sperms, sperm activity, and whether sperms have deformity may be identified. Particularly, after improving the state of sperms, it is possible to try to get pregnant.

In analyzing semen, the subdivision pattern may be used as shown in FIGS. 6(a) and 6(b). By determining the number, activity, and the form of sperms positioned within the grid pattern formed in the middle of FIGS. 6(a) and (b), the sperm characteristic is easily analyzed. More specifically, all sperms distributed throughout the obtained image are not analyzed by the user terminal 100 or the medical institution system 200, sampling analysis may be performed on targets that are sperms in the region delimited by the subdivision pattern. According to the present invention, quick analysis is possible, and pattern processing load of the medical institution system 200 or of the user terminal 100 may be reduced with more detailed analysis.

As an example of the subdivision pattern, although the grid form is shown in FIGS. 5 and 6, the pattern may be formed in various shapes, such as an oval, a circle, etc., and an arbitrary position may be specified as well as the center of the screen. Also, the image is checked, and the subdivision pattern is applied to the position with high density, whereby the analysis is conducted. The present invention is not limited thereto.

In order to analyze the sperm characteristic in the user terminal 100 or the medical institution system 200 of the present invention, a similar method to the above-described saliva analysis is performed. First, the user terminal 100 photographs the semen deposited on the sheet unit 5 to obtain the test image. The user terminal 100 or the medical institution system 200 computes the number of sperms, travel distances of particular sperms, the average of the travel distances, whether to rotate, the rotation speed, and the rotation radius identified within a particular region in the test image. Here, the user terminal 100 or the medical institution system 200 may obtain several test images for use, or may obtain the test image in the form of a video. Here, particular sperms in the image are randomly selected and the sperm characteristic of the randomly selected sperms is determined, whereby the state of sperms is identified. Also, the selection of multiple sperms for sampling may enhance accuracy of the analysis of the sperm characteristic.

Also, the user terminal 100 or the medical institution system 200 selects some sperms in the test image, the image of the selected sperms is processed in a clear pattern or in a black-and-white image, and then the result is compared with a pre-stored image or with a predetermined condition whether to have a shape corresponding thereto, whereby whether sperm is malformed is determined. For example, whether sperms in the test image have a normal form may be identified by determining the number of end portions (tail shape) per head (round part), whether the end portion is bent, and malformation of the head.

Particularly, in this process, in order to facilitate selection and sampling of sperms, the subdivision pattern is applied as shown in FIG. 6, and the sperm characteristic may be analyzed by targeting the sperms positioned in the region defined by the subdivision pattern among sperms in the test image. Thus, selection of sperms for sampling may be easy, consistency of the analysis information may be maintained, and the load of the user terminal 100 or of the medical institution system 200 due to the analysis may be reduced, whereby quick analysis is possible.

Also, as shown in FIG. 10(c), the vaginal discharge is observed to determine the presence of disease symptoms, such as bacterial vaginosis, vaginitis, and whether to perform diagnosis. Specifically, when the vaginal discharge is observed by using a testing device, only vaginal mucosa cells are observed for a normal case. However, as shown in FIG. 10(c), when a disease caused by bacterial infection occurs, substances such as those shown as black spots in FIG. 10(c) are identified. FIG. 10(c) shows the case in which secretion of anti-bacterial substances inside the vagina decreased resulting in an increase of vaginal acidity and overgrowth of anaerobic bacteria. The overgrown anaerobic bacteria is adsorbed on the surface of the vaginal mucosa cell and the vaginal mucosa cells are covered with black spots, and the symptom is identified by the testing device. As a result, according to the present invention, body secretion such as the saliva, the vaginal discharge, and the semen of man is photographed in a magnified manner and an image analysis and a naked-eye analysis are conducted such that various states, such as the presence of a disease, the name of the disease, and the ovulation period, are possibly determined.

FIG. 7 is a flowchart illustrating a body fluid state diagnostic method according to the present invention.

Referring to FIG. 7, the body fluid state diagnostic method according to the present invention may include: a body fluid reception step S10, an image acquisition step S20, an analysis information generation step S30, an output step S40, a transmission step S50, a storage step S60, and a diagnosis step S70. The storage step S60 or the diagnosis step S70 may include a regeneration step S65.

At the body fluid reception step S10, body fluid of the user who wants to have a test is received. At the body fluid reception step S10, the user deposits the body fluid extracted from the user's body on the base 5 of the testing tool. At the body fluid reception step S10, the body fluid may be dried for a particular time, and particularly, for confirmation of the ovulation period, the body fluid is dried and the user waits until the crystal is formed.

At the image acquisition step S20, the user terminal 100 photographs the body fluid of the user placed on the testing tool 1, and obtains the test image. To this end, at the image acquisition step S20, the testing tool 1 may be coupled to the user terminal 100, and the image of the base 5 focused on a camera lens of the user terminal 100 by the coupling is obtained. At the image acquisition step S20, the testing tool 1 magnifies the image of the base 5 by a predetermined magnification or a magnification adjusted by the user, and the user terminal obtains the image of the base 5, specifically, the image of the body fluid deposited on the base 5, which is enlarged with the magnification by the testing tool 1 in a still image or a video format. In this process, when the magnification provided by the testing tool 1 is insufficient, an optical zoom function, etc. of the camera of the user terminal 100 may be used to enlarge the image of the body fluid, but the present invention is not limited thereto.

At the analysis information generation step S30, the body fluid is analyzed by conducting a pattern analysis on the image obtained by the user terminal 100. At the analysis information generation step S30, the image obtained by the user terminal 100 is transformed into a simplified image, e.g., a black-and-white image for easy data processing. This is performed to clarify the form of the crystal or the form of the sperm formed from the body fluid to facilitate distinguishment the background and the crustal or the background and the sperm. Here, since conventional various methods may be used as the image processing method, the detailed description thereof will be omitted. After the image is processed, the user terminal 100 performs a process of analyzing the form of the crystal or the form of the sperm and the characteristic from the image. Thus, the user terminal analyzes the crystal characteristic and the sperm characteristic, such as the form of the crystal, density of the crystal, the length of the line formed by the crystal, the form of the sperm, straightness of the sperm, the speed of the sperm, and whether the sperm rotates.

In this process, the user terminal 100 may limit the range of the image on which the analysis is conducted by the lens of the testing tool 1 or the subdivision pattern applied in obtaining the image, and may conduct the analysis within the limited range. After the analysis, the user terminal 100 generates the analysis information containing the analysis result, such as whether to be in the non-ovulation period, the transition period, or the ovulation period, whether the sperm is normal. Here, the generated analysis information may refer to information generated in the form that may be processed and output by the user terminal 100 or the medical institution system 200.

At the output step S40, the generated analysis information may be output via the output unit of the user terminal 100, or may be transmitted in the form specified by the user, such as an e-mail, and a message. At the output step S40, the user may know the examination result of the user's body fluid.

At the transmission step S50, the user terminal 100 transmits the test image and the analysis information to the medical institution system 200. The present invention may provide the body fluid state diagnostic system and method that enables the user to check the user's state by using the user terminal 100, but connection to the specialized medical institution is possible. In this case, the step after the transmission step S50 may be performed for connection to the medical institution.

In the meantime, at the transmission step S50, the user terminal 100 may transmit only the test image to the medical institution system, but the present invention is not limited thereto. The medical institution system 200 may be a system operated by a specialized medical institution, such as a hospital, or may be a company or an institution that maintains the analysis information when connecting the medical institution and the user. However, in the present invention, the description is made assuming that it belongs to the specialized medical institution. To this end, the user terminal 100 may access the medical institution system 200 via a mobile network or a wired or wireless data network, or may access medical institution system 200 via a network service such as the Internet to transmit the test image and the analysis information with membership information.

At the storage step S60, the medical institution system 200 which has received the test image and the analysis information from the user terminal 100 stores the test image and the analysis information by being matched with the corresponding membership according to the membership information received from the user terminal 100 or membership information distinguished by using information of the user terminal 100.

At the regeneration step S65, the medical institution system 200 reanalyzes the received test image. The regeneration step S65 is selectively performed to reconfirm the analysis information generated by the user terminal 100. Alternatively, in the present invention, the analysis information is generated by the user terminal 100, but an analysis which is difficult to be conducted in the user terminal 100, e.g., when the performance of the user terminal 100 is poor, may be conducted in the medical institution system 200, and then the analysis information may be transmitted to the user terminal 100.

At the diagnosis step S70, the analysis information generated by the user terminal 100 or the medical institution system 200 is provided to a medical specialist, e.g., a specialist, who carries a terminal connected with the medical institution system 200. Furthermore, at the diagnosis step S70, the diagnostic result from the medical specialist may be transmitted to the user terminal 100 by the medical institution system 200, and the user terminal 100 may output the result to the user, but the present invention is not limited thereto.

While the exemplary embodiments of the invention have been described above, the embodiments are only examples of the invention, and it will be understood by those skilled in the art that the invention can be modified in various forms without departing from the technical spirit of the invention. Therefore, modifications of the present invention are appreciated as included in the scope and spirit of the invention, and the protection scope of the present invention will be defined by the accompanying claims.

### INDUSTRIAL APPLICABILITY

According to the present invention, the body fluid state diagnostic system and state diagnostic method using the same enable the user to perform self-diagnosis and identify the diagnostic result as well as enable reconfirmation and a diagnosis through the specialized medical institution, whereby the body state of the user is managed in connection to specialized medical technologies of the specialized medical institution.

1. A body fluid state diagnostic system comprising:
a testing tool having a base on which a test target that is body fluid of a user is deposited; and
a user terminal obtaining a test image of the body fluid deposited on the base by photographing the base of the testing tool, generating analysis information by analyzing the test image, and outputting the analysis information depending on selection of the user.

2. The system of claim 1, further comprising:
a medical institution system receiving the test image or the analysis information via a network in connection to the user terminal, storing the test image or the analysis information by matching with information of the user which is pre-stored, and performing a reanalysis by using the test image to determine validity of the analysis information or to provide an analysis result to a medical specialist.

3. The system of claim 2, wherein the body fluid is saliva, semen, or vaginal discharge of the user.

4. The system of claim 3, wherein the user terminal performs a pattern analysis of a crystalline characteristic including at least one of density, a direction, and a length of a crystal formed from the body fluid in the test image.

5. The system of claim 3, wherein the user terminal performs a pattern analysis of a sperm characteristic including at least one of activity, density, and malformation of a sperm in the body fluid.

6. The system of claim 5, wherein the activity of the sperm includes at least one of straightness, a straight-line distance, and a rotational tendency of the sperm.

7. The system of claim 4, wherein the testing tool or the user terminal or the medical institution system uses a subdivision pattern limiting a range of the image to determine the crystalline characteristic or the sperm characteristic.

8. The system of claim 7, wherein the user terminal or the medical institution system performs the pattern analysis on a target which is the crystal formed within the subdivision pattern or the sperm positioned within the subdivision pattern.

9. The system of claim 2, wherein the testing tool is detachably attached to the user terminal.

10. The system of claim 2, wherein the testing tool includes an enlargement unit providing an enlarged image of the body fluid deposited on the base.

11. The system of claim 2, wherein the user terminal photographs the body fluid provided by the testing tool in a magnified manner.

12. A body fluid state diagnostic method comprising:
a body fluid reception step at which body fluid of a user is deposited on a base of a testing tool;
an image acquisition step at which a user terminal photographs the base and obtains a test image of the body fluid;
an analysis information generation step at which the user terminal analyzes the test image to generate analysis information; and
an output step at which the user terminal outputs the analysis information.

13. The method of claim 12, further comprising:
a transmission step at which the user terminal is connected to a medical institution system of a specialized medical institution via a network and the test image or the analysis information is transmitted to the medical institution system;
a storage step at which the medical institution system stores the analysis information or the test image by matching with information of the user; and
a diagnosis step at which the medical institution system transmits the analysis information to a medical specialist of the specialized medical institution.

14. The method of claim 13, wherein the storage step or the diagnosis step includes a regeneration step at which the medical institution system performs a reanalysis of the test image to regenerate the analysis information.

15. The method of claim 14, wherein the body fluid is saliva, semen, or vaginal discharge of the user.

16. The method of claim 15, wherein at the analysis information generation step or the regeneration step, the user terminal or the medical institution system performs, by using the test image, a pattern analysis of a crystalline characteristic including at least one of density, a direction, and a length of a crystal formed from the body fluid.

17. The method of claim 15, wherein at the analysis information generation step or the regeneration step, the user terminal or the medical institution system performs a pattern analysis of a sperm characteristic including at least one of straightness, a straight-line distance, and a rotational tendency of a sperm.

18. The method of claim 12, wherein at one of the image acquisition step, the analysis information generation step, and the regeneration step, the testing tool or the user terminal or the medical institution system uses a subdivision pattern limiting a range of the image to determine the crystalline characteristic or the sperm characteristic of the body fluid.

19. The method of claim 18, wherein at one of the image acquisition step, the analysis information generation step, and the regeneration step, the pattern analysis is performed on a target which is the crystal formed within the subdivision pattern or the sperm positioned within the subdivision pattern.

20. The method of claim 18, wherein at the image acquisition step, the user terminal photographs the body fluid in a magnified manner.
